(19) United States
(12) Patent Application Publication
(10) Pub. No.: US 2019/0096525 A1
(43) Pub. Date: Mar. 28, 2019

(54) PATIENT DATA MANAGEMENT SYSTEM

(71) Applicant: Owned Outcomes Inc., Las Vegas, NV (US)

(72) Inventors: Mariusz Ferenc, Bialystok (PL); Wojtek Kozlowski, Bialystok (PL); Krupa Srinivas, Las Vegas, NV (US); Huzaifa Sial, Las Vegas, NV (US); Anita Pramoda, Las Vegas, NV (US)

(21) Appl. No.: 15/988,785

(22) Filed: May 24, 2018

Related U.S. Application Data
(60) Provisional application No. 62/563,569, filed on Sep. 26, 2017.

(51) Int. Cl. G16H 50/20; G06N 3/08

(52) U.S. Cl. CPC G16H 50/20; G06N 3/08

(57) ABSTRACT

A patient data management (“PDM”) system is disclosed herein. The PDM can provide doctors with an efficient and accurate means to extract medical diagnostic and treatment information from multi-perspective time based medical data. Further, the PDM provides a means to reduce computer processing time when training a neural network using medical data. In one aspect, a PDM system includes a preprocessor. The preprocessor receives patient data from a computer interface. In one non-limiting example, the preprocessor uses machine learning to extract patterns (“features”) from the data. The preprocessor formats the extracted features into a multidimensional tensor. In one non-limiting example, the PDM system includes a convolutional neural network (“CNN”). The preprocessor provides the tensor to the CNN. The CNN processes the tensor and extracts diagnostic and treatment information.

### CROSS-REFERENCE TO RELATED APPLICATIONS

The present application claims priority to U.S. Provisional Application No. 62/563,569 entitled “PATIENT DATA MANAGEMENT SYSTEM” filed Sep. 26, 2017, and hereby expressly incorporated by reference herein.

### TECHNICAL FIELD

The present disclosure relates to systems and methods for reducing computer processing time when training a neural network using medical data. The present disclosure further relates to systems and methods for improving the accuracy of neural networks trained to generate medical diagnostic and treatment information.

### BACKGROUND

There is a need for systems and methods that can efficiently and accurately extract medical diagnostic and treatment information from multi-perspective time based medical data. During the course of a patient's treatment, doctors collect large amounts of medical data from multiple sources. This medical data is often stored in a variety of formats such as image, audio, numerical, and textual. Each format comprises multiple data points. Each data point may be associated with a date and time. This raw medical data is noisy and often contains vast amounts of irrelevant and redundant information.

The problem with this medical data is that, when viewed as whole over the course of an illness (“episode”), doctors often miss meaningful patterns (“features”) in the data because of the data's complexity. Relationships between medical data sources are often hidden deep in the data, across multiple data sources, and over long time frames. Seemingly insignificant data may, under certain circumstances, be a dominant feature that is affecting the course of a patient's illness.

### SUMMARY OF THE INVENTION

The present disclosure relates to systems and methods for reducing computer processing time when training a neural network using medical data. The present disclosure further relates to systems and methods for improving the accuracy of neural networks trained to generate medical diagnostic and treatment information.

One embodiment is an electronic system for determining features of medical data (“feature selection”), the system including a processor comprising instructions that when executed perform the following method: receiving, patient medical data; converting the received patient medical data into a plurality of tensors; extracting from deep canonical correlation, features of the medical data shared across the tensors; and analyzing the features from the medical data using a neural network to discover patterns in the medical data.

Another embodiment includes a system for improving the accuracy of a convolutional neural network, the system comprising: a preprocessor configured to: receive patient medical data; convert the received patient medical data into a plurality of tensors; extract, from deep canonical correlation, features of the medical data shared across the tensors wherein the shared features are represented as a tensor; and analyze the features from the medical data using a neural network to discover patters in the medical data.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 is a flowchart illustrative of one embodiment of a patient data management (“PDM”) system integrated into a hospital's workflow.

FIG. 2 is a flowchart illustrating an example process for a preprocessor of the PDM system of FIG. 1.

FIG. 3 illustrates an example representation of tensor slices.

FIG. 4 illustrates an example representation of clinical episode sequence data.

FIG. 5 illustrates an example representation of tensor slices.

FIG. 6 illustrates an example representation of a tensor created by the PDM system of FIG. 1.

FIG. 7 illustrates an example representation of medical data generated during a clinical episode, stored in an array of vectors, where each vector represents a single data source.

FIG. 8 illustrates an example representation of the tensor created by the PDM system of FIG. 1.

FIG. 9 illustrates an example representation of a deep canonical correlation analysis.

FIG. 10 illustrates and example representation of a convolutional neural network (“CNN”).

### DETAILED DESCRIPTION

The present disclosure relates to systems and methods for reducing computer processing time when training a neural network using medical data. The present disclosure further relates to systems and methods for improving the accuracy of neural networks trained to generate medical diagnostic and treatment information.

The systems and methods described herein may process input from either a group of patients or from a single patient. Additionally, the systems and methods may use patient data to create diagnostic information regarding an entire group of patients, a subset of the group of patients, or a single patient.

For example, a patient visits a doctor complaining of nausea, fatigue, and indigestion. The doctor measures the patient's pulse, blood pressure, temperature, and weight. The doctor then decides to order an electrocardiogram test in order to rule out heart disease. However, the electrocardiogram test results come back as inconclusive. The following week, the patient returns to the doctor complaining of the same symptoms. This time the doctor orders a computed tomography test for the patient. Again the results come back as inconclusive. Less than a week later, the same patient returns to the doctor with the same symptoms. This time, the doctor decides to use a Patient Data Management (PDM) system to aid in diagnosing the patient's disease.

The doctor begins the process by uploading, through a computer interface to a preprocessor, all of the patient's test data, as well as all historical medical data on file for that patient. This historical medical data may include a variety of data gathered during earlier treatments, including the diagnosis codes from each prior visit to a physician. A listing of prior interventions, medicines, and lab results may also be uploaded into the system. In addition to these medical-related data points, the doctor may also upload additional data on the patient, for example socio-demographic data relating to the patient that was gathered previously. The preprocessor then takes multi-perspective time-based patient data and forms it into a tensor.

As used herein, a tensor is a mathematical object that is analogous to, but more general than, a vector. In some embodiments, a tensor may be represented by an array of components that are functions of the coordinates of a space. A tensor can be represented as an organized multidimensional array of numerical values or scalars. For example, a one dimensional tensor can be a vector, a two dimensional tensor can be a matrix, and three dimensional tensor can be a cube of scalars.

The preprocessor applies the methods and steps described below and then feeds the tensor to a convolutional neural network (CNN). In one embodiment, the CNN has previously been trained to classify the tensor data according to disease type. After processing the tensor, the CNN may output a specific recommendation to the doctor based on the uploaded and pre-processed data. In the example above, the CNN may analyze the patient data and output a high probability that based on the patient's medical data, the patient likely has heart disease. Of course, this is just one example of the type of medical output that the system may provide to the doctor. The figure descriptions below describe the PDM system in greater detail.

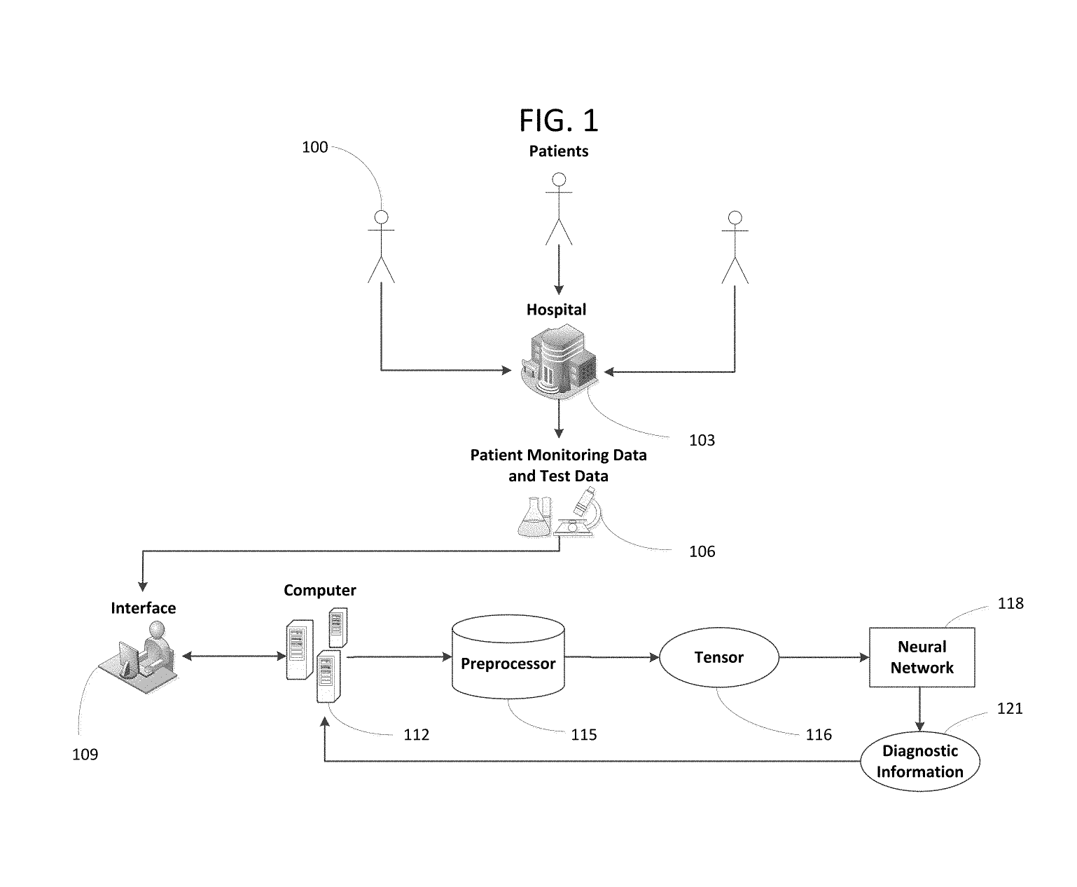

FIG. 1 is a flowchart illustrative of one embodiment of a PDM system integrated into a hospital's workflow. Hospital patients 100, visit a hospital 103, to get diagnosis and treatment information for health issues. While at the hospital 103, a doctor collects patient monitoring data and test data 106, from the patients 100. The doctor enters the test data 106, into an interface 109, associated with a computer 112.

The computer 112, processes the patient monitoring data and test data 106, using a preprocessor 115. In one embodiment, the preprocessor 115 converts the data 106 into a three dimensional tensor 116. In some embodiments, the tensor 116, may have additional dimensions, for example, four or five dimensions. Next, the preprocessor 115, feeds the tensor 116, to a neural network 118. In some embodiments, the neural network 118, may comprise a convolutional neural network. In other embodiments the neural network 118, may comprise a different type of network such as a long or short term memory network.

The neural network 118, processes the tensor 116, to extract features. The neural network 118, may use these features to create diagnostic information 121, such as disease categorization, medical treatment predictions, and the like. The computer 112, transmits the diagnostic information 121, to the interface 109, for the doctor to read and relay to the patient 100.

In some embodiments, the computer 112, may be located at the hospital 103 or remotely in the cloud. In some embodiments the computer 112, may comprise a virtual server. In other embodiments, the computer 112, may comprise a handheld device and the like.

In some embodiments, the preprocessor 115, may be comprised of electronic circuits. In other embodiments, the preprocessor 115, may be comprised of source code performed by a processor located in the computer 112. In yet other embodiments, the preprocessor 115, may be comprised of a combination of electronic circuits and source code.

In some embodiments the neural network 118, may be comprised of electronic circuits. In other embodiments, the neural network 118, may be comprised of source code performed by a processor on the computer 112. In yet other embodiments, the neural network 118, may be comprised of a combination of electronic circuits and source code.

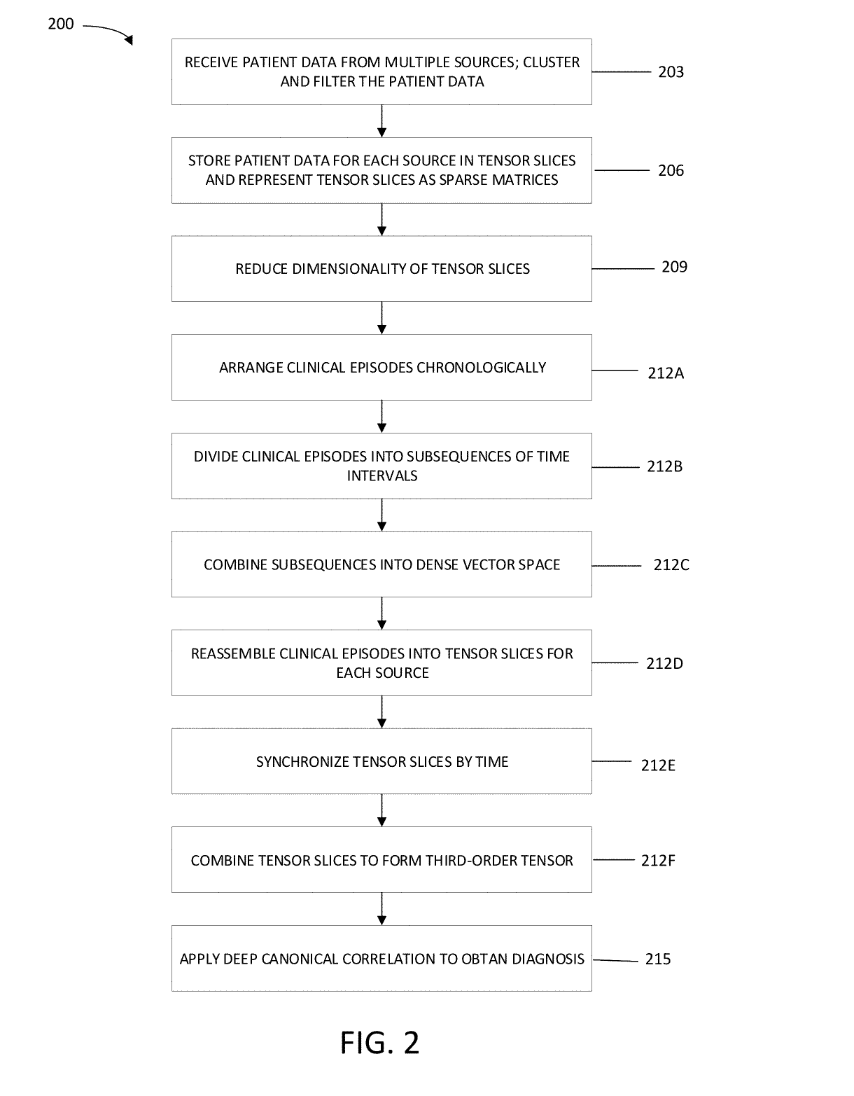

FIG. 2 is a flowchart 200 illustrating an embodiment of a process running in the preprocessor 115, of the PDM system of FIG. 1. The process begins with step 203, where the preprocessor 115 receives patient data from the interface 109. The patient data can be gathered from a number of sources, including electronic medical records, electronic health records, procedure, resource and billing codes. The preprocessor 115 can then apply space clustering to the patient data 106, thereby separating the data into disease and non-disease related data clusters. This enables the preprocessor 115 to filter out the non-disease related data clusters when forming the final tensor. By applying space clustering to the patient data 106, the problem of heterogeneity of a given dataset is reduced due to the fact that, for instance, each Diagnosis has primarily its own vector space. The preprocessor 115 may apply space clustering using either a regular clustering algorithm such as Expected Maximization using Fisher criteria or via ontology coding such as the Medicare Severity-Diagnosis Related Group system.

The process 200 next moves to step 206, where the preprocessor 115 allocates storage space for the data of step 203. The preprocessor 115 stores each source of clinical data into separate tensor slices 116. For example, each source of data may be from an electronic medical record, electronic health record, MAR etc.) The tensor slices 116 may be represented as sparse matrices to save storage space. In this example, each tensor slice 116 comprises multiple one dimensional arrays (“vectors”) and each vector represents a clinical episode. A clinical episode is one clinical activity, such as the results of a checkup, a prescription, a surgical outcome, a diagnosis, socio-demographics, regional climate information, etc. In other words, a clinical episode can be any information that may be useful in the diagnostic process. In this embodiment, each vector comprises clinical data points.

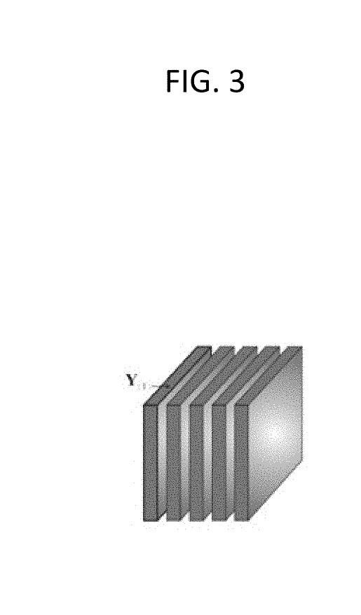

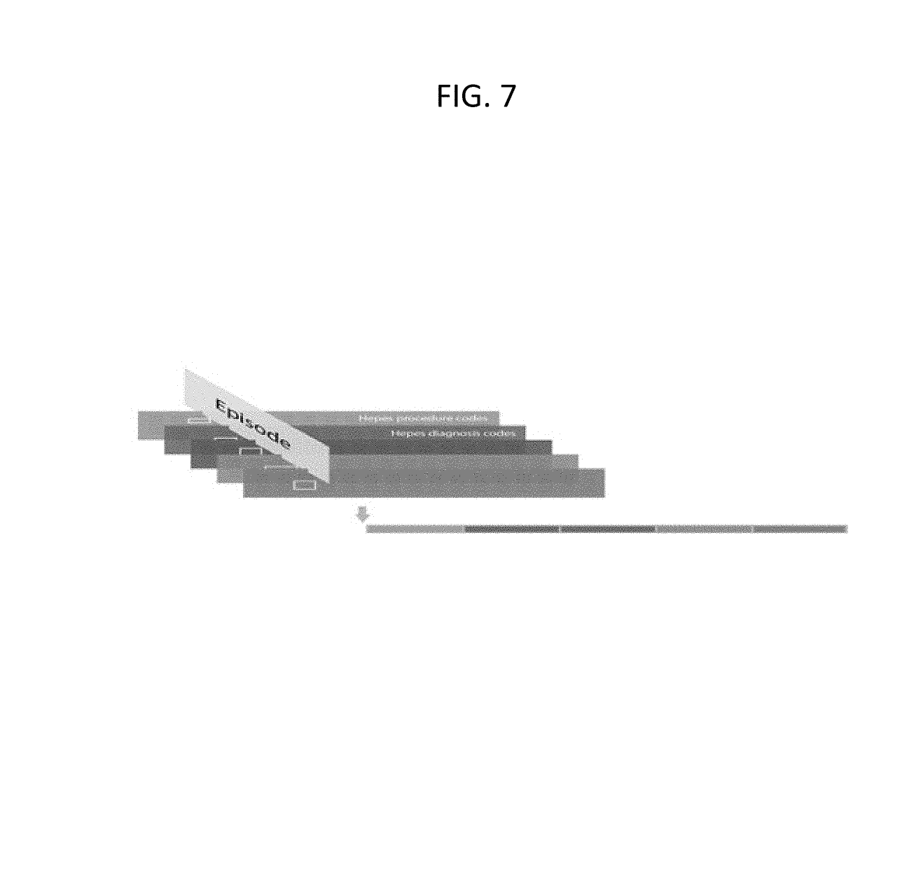

In one embodiment, each tensor slice 116 may be defined as: Xi∈ [symbol] m×n, where X is a matrix, m is the space dimension, n is the number of clinical episode instances, and i={0, 1, . . . , k} is a given vector space's number. The result of step 206 is a collection of tensor slices 116 represented as sparse matrices defined as Y={X₁, . . . , Xₖ}. FIG. 3 depicts an example sparse matrix Y which includes tensor slices 116. This assembly of higher order sparse tensors can include sparse tensors from simultaneous spaces. Each tensor slice includes vector representations of clinical episodes received from a particular source. FIG. 7 depicts another example of these tensor slices 116 with an additional depiction of an instance of a clinical episode from a mathematical point of view (i.e., an intersection of a given sparse tensor.

After allocating storage space and storing each source of clinical data into separate tensor slices 116 at step 206, the preprocessor 115 moves to step 209 and reduces the complexity of the data by reducing the dimensionality of the tensor slices 116 determined in step 206. In this example, each tensor slice 116 is made up of unknown low-level features. Applying dimensionality reduction locates features within the tensor slices 116 and removes the irrelevant and/or redundant features. The result is a compressed representation of the original tensor slices 116. Step 209 may decrease the total number of convolutions that will be created in the CNN thereby saving processing power and time.

The preprocessor 115 may dimensionally reduce the tensor slices 116 using a variety a machine learning techniques including, but not limited to, Singular Value Decomposition (SVD), Non-negative Matrix Factorization, Tensor Matrix Factorization, and Sparse Auto-encoding. SVD allows for significant dimensionality reduction while preserving meaningful information. When using SVD, the PDM can reduce each tensor slice 116 by the same number of dimensions. Using a sparse auto encoder can also produce similar dimensionality reduction results.

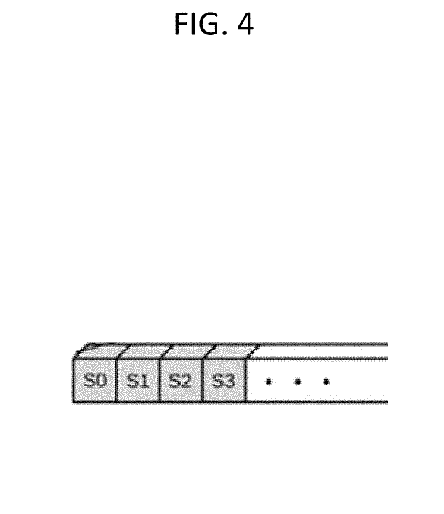

The process 200 next moves to step 212A, where the preprocessor 115 orders each vector (i.e., clinical episode) in each tensor slice 116 by time of occurrence (i.e., chronologically). The purpose of this step is to represent each clinical episode as a collection of codes that occur on the axis of time. The results of this stage are ordered time sequence pairs (“episode sequences”). An episode sequence can be formatted as {(c1,t1), (c2,t2), . . . , (ch,th)} where “ci” is denoted as a clinical code and “ti” is the time value preserving order: t1<t2<. . . <th. After ordering the episode vectors into sequences, the preprocessor 115 moves to step 212B and divides the episode sequences into uniform subsequences of a pre-defined interval (e.g. 1-3 days). Each subsequence is part of a whole episode sequence and includes all clinical episodes that appear in a given time range within the tensor. FIG. 4 depicts a representation of the uniform subsequences S0, S1, S2, S3, etc. of step 212B. For example, each subsequence can represent a 3 day interval, with S0 occurring before S1, etc. Each subsequence includes episode sequences (i.e., chronologically ordered clinical episodes) that occurred within that 3 day interval. By dividing clinical episodes into subsequences, we can assume linearity and regard each subsequence as a linear subspace. Step 212B of FIG. 2 addresses the issue of medical data time resolution varying depending on the source and type of data. For example, a patient's blood pressure data might have been collected daily while the patient's blood test data was only collected weekly. Moreover, diseases and treatments can progress and develop overtime. Each disease can have a unique progression, such as periods where new symptoms appear or where comorbidities develop (e.g. hypertension appearing at a specific point in the diabetic disease). Likewise, the causes of a fever can be different depending on whether the fever occurred before administering medication or just after. Thus, monitoring such dynamic processes overtime can be a decisive factor in treatment.

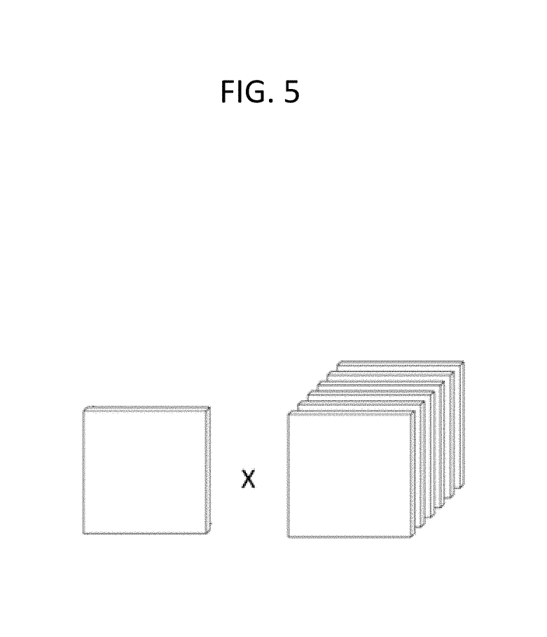

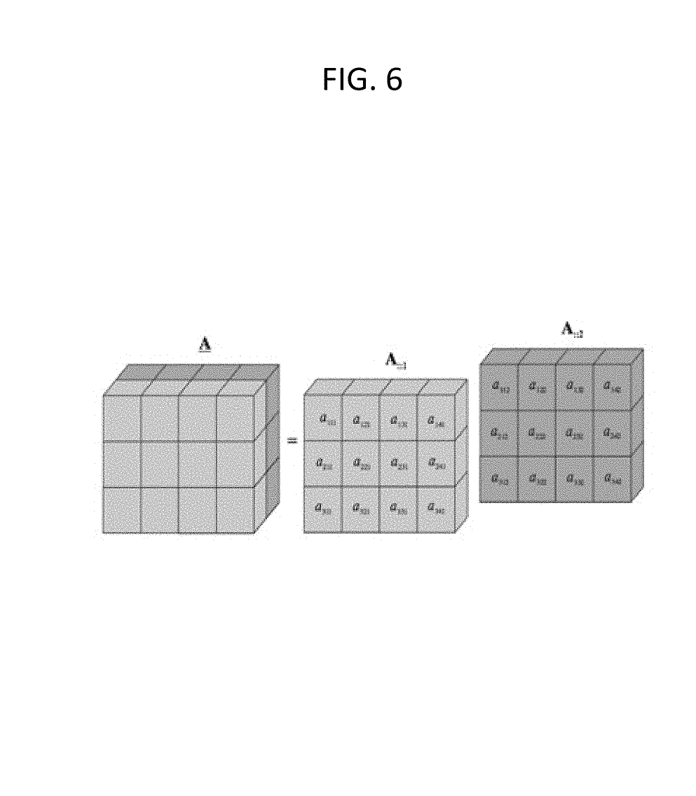

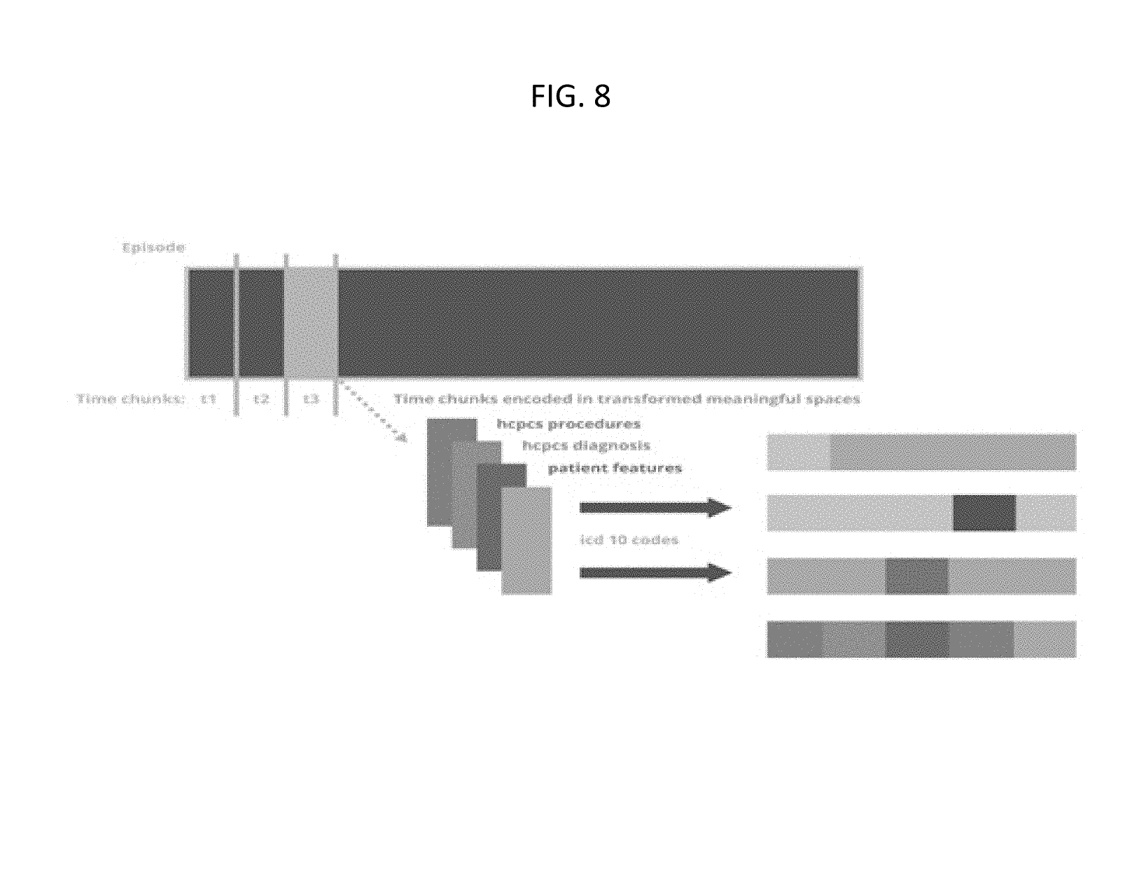

After creating the subsequences (S0, S1, S2, S3, etc.), the process 200 moves to step 212C where the preprocessor 115 combines the subsequences into a dense vector space of k dimensions where v∈ [symbol] , using the resulting parameters from the matrix factorization of step 206. Similarly, if a sparse auto-encoder was used in step 206, then subsequence vectors should be transformed by the sparse auto-encoder network. Next, in step 212D, the preprocessor 115 re-assembles the episode vectors back into individual tensor slices 116 for each data source. The re-assembled tensor slices 116 remain in chronological order and are in the form of tensors with increased dimensionality (e.g, +1 higher dimensionality) FIG. 5 illustrates an example representation of these tensor slices. Using the dense vectors created in step 212C, each tensor slice can be represented as a matrix of Mᵢ={v₁, v₂, . . . , vₙ}, where v1, v2, . . . vn are dense vectors. In step 212E, the preprocessor 115 synchronizes the tensor slices by time to have the effect of consistency over time (i.e. keep time topology). In step 212F, the preprocessor 115 combines the tensor slices to form a third order tensor. FIG. 6 depicts an example of the assembled third order tensor 616. Using the third order tensor 616, data can be represented in both a latitudinal approach (i.e. from the perspective of various spaces, including diagnostic, procedural, laboratory, or from the perspective of the main disease, and simultaneously from the perspective of comorbidities) and a longitudinal approach (i.e., observing changes in the timeline of all spaces at once. The third order tensor can be defined as T={M₁, M₂, . . . , Mm}. FIG. 8 depicts a representative extension for the time factor. In FIG. 8, the process of separating “time chunks” 810 on a timeline for a given clinical episode is shown. Further, the production of dense vectors for each time chunk 810 is represented within various spaces 820 (e.g., spaces for diagnoses, procedures, etc.). In this manner, the resulting collection of vectors can next generate a tensor, as represented in the bottom right of FIG. 8.

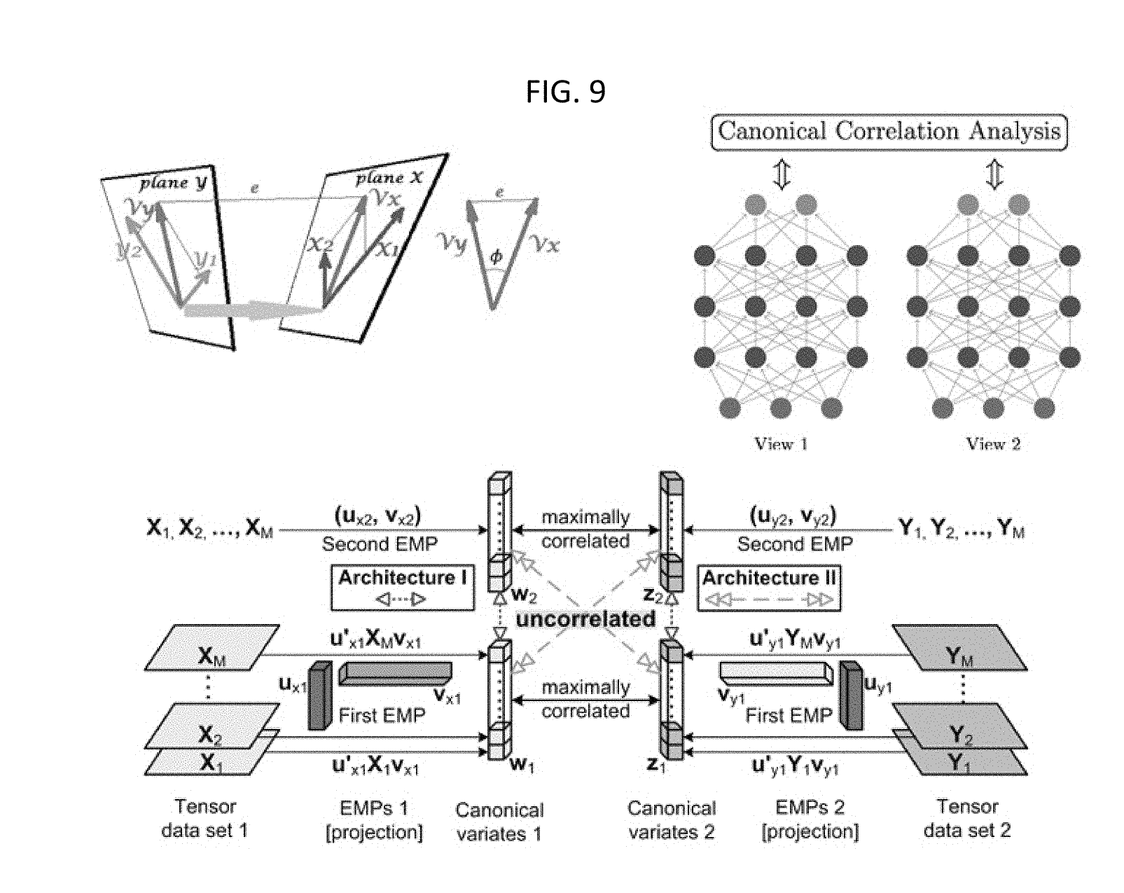

The process 200 next moves to step 215, where the preprocessor 115 applies deep canonical correlation (DCC) to the third order tensor tensor 616 of step 212F. The goal of this step is to find variables shared across the tensor slices. Because each tensor slice 116 represents a different medical data source, each tensor slice 116 comprises data that, on its face, is dissimilar from the others tensor slices 116. For example, one tensor slice 116 might contain breathing data while another could contain blood platelet count. DCC can find hidden variables shared by these two tensors slices 116 and then maximize that correlation. Once DCC is applied, the resulting tensor is a third order tensor that is organized using canonical coordinates. The tensor also includes a time feature making it very effective in representing disease and treatment progression. In some embodiments, the tensor of step 212F, may have more than three dimensions. FIG. 9 illustrates an example representation of a deep canonical correlation analysis. Each vector space used, is transformed by various types of transformations in solation from other simultaneous spaces to move to the dense vector space. As a result, there is a desynchronization of features. It can be beneficial to synchronize features using a mechanism such as Canonical Correlation Analysis.

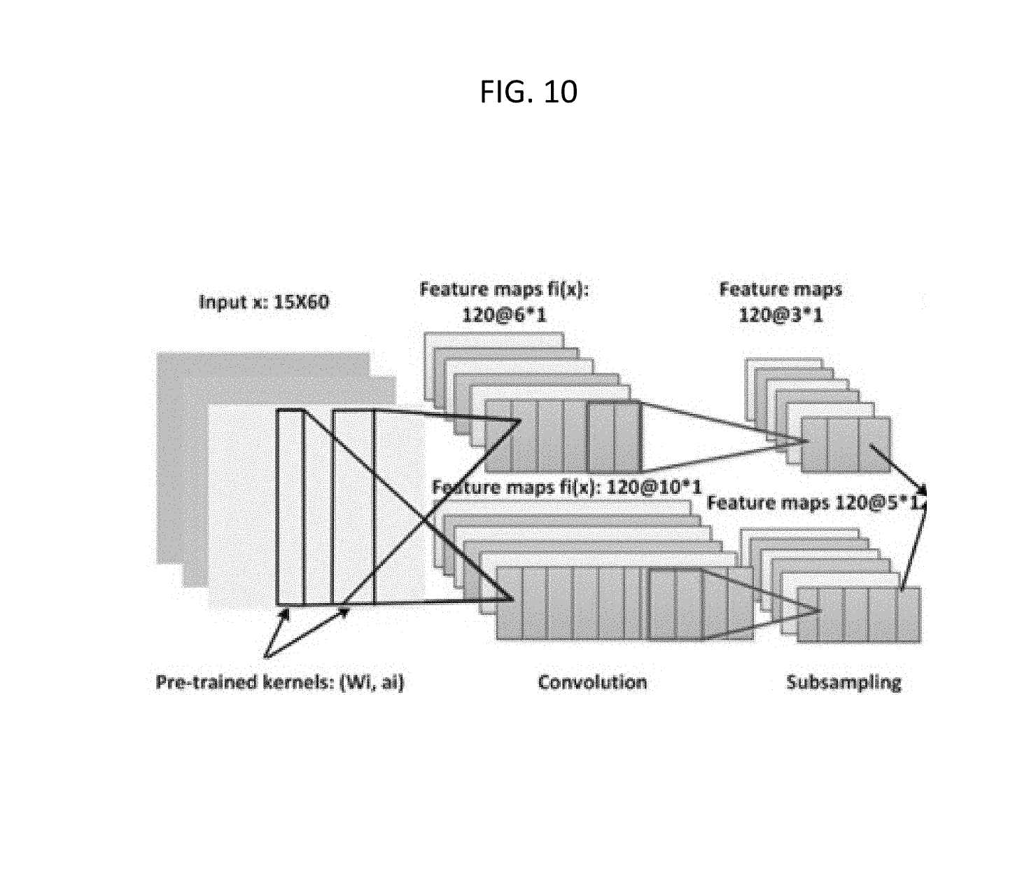

In some embodiments, the preprocessor 115 feeds the tensor of step 215, to a CNN for analysis and output to the interface 109 of FIG. 1. CNN performance can be improved by applying variable-size convolution filters to extract variable-range features of clinical episodes. FIG. 10 depicts an example of variable-size convolution filters. CNN performance can additionally be increased by applying mutual learning and kernel pre-training. By using this process, hidden correlations between the original patient data can be found and used to determine diagnoses and treatment options for the patient.

To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The various illustrative logical blocks, modules, and circuits described in connection with the implementations disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or process described in connection with the implementations disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of non-transitory storage medium known in the art. An exemplary computer-readable storage medium is coupled to the processor such the processor can read information from, and write information to, the computer-readable storage medium. In the alternative, the storage medium may be integral to the processor. The processor and the storage medium may reside in an ASIC. The ASIC may reside in a user terminal, server, or other device. In the alternative, the processor and the storage medium may reside as discrete components in a user terminal, server, or other device.

Headings are included herein for reference and to aid in locating various sections. These headings are not intended to limit the scope of the concepts described with respect thereto. Such concepts may have applicability throughout the entire specification.

The previous description of the disclosed implementations is provided to enable any person skilled in the art to make or use the present invention. Various modifications to these implementations will be readily apparent to those skilled in the art, and the generic principles defined herein may be applied to other implementations without departing from the spirit or scope of the invention. Thus, the present invention is not intended to be limited to the implementations shown herein but is to be accorded the widest scope consistent with the principles and novel features disclosed herein.

What is claimed is:

1. An electronic system for determining features of medical data, the system comprising a processor comprising instructions that when executed perform the following method:
receiving, patient medical data;
converting the received patient medical data into a plurality of tensors;
extracting from deep canonical correlation, features of the medical data shared across the tensors; and
analyzing the features from the medical data using a neural network to discover patterns in the medical data.

2. The system of claim 1, wherein the patient medical data comprises a plurality of data types, the data types comprising a plurality of data points.

3. The system of claim 2, wherein the method further comprises separating the data for each data type, into a plurality of data clusters, wherein the data clusters comprise disease related data points represented as one dimensional vectors, each vector representing a clinical episode.

4. The system of claim 3, wherein the method further comprises combining, the vectors for the plurality data types into a plurality of tensor slices, wherein each tensor slice is represented as a sparse matrix.

5. The system of claim 4, wherein the method further comprises compressing, the plurality of tensor slices.

6. The system of claim 5, wherein the method further comprises arranging, by time of occurrence, each tensor slice's data points.

7. The system of claim 6, wherein the method further comprises synchronizing, by time, the plurality of tensor slices.

8. The system of claim 5, wherein the preprocessor compresses the tensor slices using singular value decomposition.

9. The system of claim 5, wherein the preprocessor compresses the tensor slices using sparse auto-encoding.

10. The system of claim 3, wherein the preprocessor creates the data clusters using expected maximization via Fisher criteria.

11. The system of claim 3, wherein the preprocessor creates the data clusters using Medicare Severity-Diagnosis Related Group encoding or other such ontology based encoding.

12. A system for improving the accuracy of a convolutional neural network, the system comprising:
a preprocessor configured to:
receive patient medical data;
convert the received patient medical data into a plurality of tensors;
extract, from deep canonical correlation, features of the medical data shared across the tensors wherein the shared features are represented as a tensor; and
analyze the features from the medical data using a neural network to discover patters in the medical data.

13. The system of claim 12, wherein the patient medical data comprises a plurality of data types, the data types comprising a plurality of data points.

14. The system of claim 13, wherein the preprocessor is further configured to separate the data for each data type, into a plurality of data clusters, wherein the data clusters comprise disease related data points represented as one dimensional vectors, each vector representing a clinical episode.

15. The system of claim 14, wherein the preprocessor is further configured to combine the vectors for the plurality data types into a plurality of tensor slices, wherein each tensor slice is represented as a sparse matrix.

16. The system of claim 15, wherein the preprocessor is further configured to compress the plurality of tensor slices.

17. The system of claim 16, wherein the preprocessor is further configured to arrange, by time of occurrence, each tensor slice's data points.

18. The system of claim 17, wherein the preprocessor is further configured to synchronize, by time, the plurality of tensor slices.

19. The system of claim 18, wherein the convolutional neural network processes the tensor using variable-size convolutional filters.

20. The system of claim 19, wherein the tensor is three dimensional.

21. The system of claim 19, wherein the convolutional neural network uses mutual learning.

22. The system of claim 19, wherein the convolutional neural network's kernels are pre-trained.
